(19) United States
(12) Patent Application Publication
(10) Pub. No.: US 2016/0175367 A1
(43) Pub. Date: Jun. 23, 2016

(54) THERAPEUTIC PARASITE PREPARATION AND METHODS OF MAKING AND USING

(71) Applicant: Donald C. Donahue, Jonesborough, TN (US)

(72) Inventor: Donald C. Donahue, Jonesborough, TN (US)

(21) Appl. No.: 14/579,506

(22) Filed: Dec. 22, 2014

(51) Int. Cl. A61K 35/62; A01K 67/033

(52) U.S. Cl. CPC A61K 35/62; A01K 67/033; A01K 2227/703; A01K 2267/0362; A01K 2267/0387

(57) ABSTRACT

A method of manufacturing a consumable parasitic composition includes: obtaining live parasite from an in vitro cell culture; and combining the live parasite with a consumable composition, the consumable composition having nutrients to maintain the live parasite, such as Hymenolepis diminuta cysticercoids (HDC) or Dipylidium caninum cysticercoids (DCC). The method includes obtaining cleansed Hymenolepis diminuta (HDO); incubating the HDO in cell growth media and fungi and bacteria so as to initiate fungal growth and bacterial growth; washing the HDO with antifungal and antibacterial; cracking the shells with hard beads (e.g., glass) in a swirled solution; filtering the cracked shells for removal of the shells; removing the HDC from cell culture media of the in vitro cell culture; and combining the HDC from the in vitro cell culture with the consumable composition.

### BACKGROUND

Generally, some human helminthic parasite compositions, preparations, methods of manufacture, and methods of use are known. That is, helminthic parasites that are specific to humans have found some uses where beneficial protocols have been explored. Some background on the human helminthic parasite compositions, manufacture, and uses can be found in the following references, which are incorporated herein by specific reference: US 20100260861; US 20100303721; U.S. Pat. No. 6,764,838; U.S. Pat. No. 7,250,173; U.S. Pat. No. 7,833,537; and J A Turton, IgE, Parasites, and Allergy, The Lancet, Sep. 25, 1976; J A Turton et al., Haematological and Immunological Responses to the Tapeworm Hymenolepis dimenuta in Man, Tropenmed. Parasit., 26 (1975) 196-200.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention relates to helminthic parasite preparations, helminthic parasite compositions, and methods of making and therapeutic methods of using the same. The helminthic parasite can be a rat-specific parasite that is particular for using a rat as a host.

In one embodiment, the invention relates to a method of producing a supplement composition comprising a helminthic parasite preparation, the method comprising the steps of: (1) raising a preparatory animal in conventional housing; (2) mixing the host preparatory animal and or parasite isolate obtained from step (1) with a carrier, such as supplement acceptable carrier. Supplement acceptable carriers are different from pharmaceutically acceptable carriers and include natural or not highly purified carriers. In one aspect, the method can exclude a pharmaceutical manufacturing method, but can encompass manufacturing any other helminthic parasite composition (e.g., not pharmaceutical) when the helminthic parasite is not in their natural state. For example, inventive compositions can include the helminthic parasite in dietary supplements (e.g., pill, capsule, liquid, solid, or the like as well as in foods, such as e.g. yogurt covered beetles, chocolate drink, or the like. The foods can include the eggs, cysticercoids, or worm or other parasite growth stage of the helminthic parasite.

In one embodiment, the invention relates to a method of producing a supplement composition comprising a helminthic parasite preparation, comprising the steps of: (1) raising a preparatory insect in conventional housing; (2) mixing the host preparatory animal and or parasite isolate obtained from step (1) with a supplement acceptable carrier.

A method for producing a pharmaceutical or nutraceutical composition comprising an rat-specific helminthic parasite in a medicinal, food, nutritional, or other comestible preparation, can include the steps of: (1) raising a preparatory animal in an environment that is not specific human pathogen-free; (2) isolating a helminthic parasite from said preparatory animal to form a helminthic parasite isolate; and (3) mixing the parasite isolate from step (2) with a carrier. The environment is not human pathogen free, and thereby may include one or more human pathogens or may include such human pathogens or the environment may be maintained without care of whether or not it is specific pathogen free. That is, the environment may be conventional and not specific pathogen free.

A method for producing a pharmaceutical or nutraceutical composition comprising a helminthic parasite preparation, can include the steps of: (1) raising a preparatory insect; (2) isolating a helminthic parasite from said preparatory insect to form a helminthic parasite isolate; and (3) mixing the parasite isolate from step (2) with a carrier, such as a carrier that is not pharmaceutically acceptable.

A method for producing a pharmaceutical or nutraceutical composition comprising a helminthic parasite preparation, can include the steps of: (1) raising a preparatory insect; (2) obtaining excrement from said preparatory insect; (3) isolating the helminthic parasite from the excrement; and (4) mixing the parasite isolate from step (3) with a carrier.

In one aspect, the carrier for the helminthic parasite preparation is not pharmaceutically acceptable, and can be food-grade. That is, the carrier is not pharmaceutical grade or otherwise highly purified or processed, but is natural or a natural product.

A cysticercoid is the larval stage of certain tapeworms, similar in appearance to a cysticercus, but having the scolex filling completely the enclosing cyst. In tapeworm infestations, cysticercoids can be seen in free form as well as enclosed by cysts in biological tissues such as the intestinal mucosa. Also referred to as a metacestode.

In one aspect, the helminthic parasite composition can be administered for a therapy. The therapies that can include administration of the helminthic parasite composition can range from immune modulation to weight loss. For example, a method of treating an individual who is obese or at risk of becoming obese can include administration of the helminthic parasite composition. The helminthic parasite can preferably include Hymenolepis diminuta (e.g., H. dimenuta), but other helminthic parasites or active variants thereof can be used, such as a nematode of Trichuris.

In one embodiment, the present invention can include compositions and methods of making the compositions that include the helminthic parasite. For example, living cysticercoids of H. diminuta can be used to established adult tapeworm in the human gut. This can be done by taking oral steroid (e.g., budesonide was used but others can work). In addition, an antihistamine can be taken (e.g., Loratadine but others can work). Both of these suppress the human immune system to allow easier colonization of the gut. In addition, a proton ion pump inhibitor can be taken to decrease the acidity of the stomach (e.g., prilosec but others can work). An H2 blocker can also work to cut down on stomach acidity (such as cimetidine or tagamet).

Cysticercoids taken by mouth without human immune suppression is also effective. This has to be repeated often though as the H. diminuta does not usually develop into reproductive adulthood. The cysticercoids can be in a helminthic parasitic composition as described herein.

In one embodiment, the present invention can include methods of obtaining living cysticercoids. In one example, the method can include: obtaining beetles having the cysticercoids and then: grinding up beetles and manually removing the cysticercoids under a microscope, or dissecting the beetles to remove them, or inserting a needle into the hemocele (abdominal cavity) of the insect (e.g., This can be done sterilely). Optionally, the beetle is first cleaned in a sterilizing solution. The insects can be raised conventionally (e.g., no need for human specific pathogen free environment) and still obtain sterile cysticercoids when extracted from the abdomen.

In one option, beetles with cysticercoids could simply be eaten. This is especially easy if a small insect is used. The beetle T. confusum is about 4 mm in length and could be placed in a capsule (with or without flour or other filler or supplement acceptable carrier) and swallowed easily. A composition can be prepared with whole, part, intact, or dismembered or ground beetle.

In one option, soluble extract of any part of the life cycle (e.g., adult, egg, or cysticercoid) could be used in the compositions and consumed in the methods. These could be extracted with water, ethanol, or oil. They could also be lyophilised. Cysticercoids could be activated in the lab prior to extraction or administration. After activation they could be grown for a period of time. It could also be possible to mimic an attacking immune system so that they produce more immune modulation substances.

Cysticercoids can be activated naturally when they pass from the stomach into the small bowel of an animal. This is also called excystation as they come out of the cyst. This is their transition phase from living in a beetle to living in a rat. It is a common lab experiment to do it artificially by manipulating the pH of their environment and adding stomach/small bowel enzymes, which can be used in the invention. The reason this may be helpful, is because only when they are activated and growing into adult tapeworms (e.g., in the small bowel) would they produce factors which effect the rat/human. So it might be advantageous to activate them, and then use the dead activated ones (or an extract) in an enteric coated capsule to deliver the intended effect in the person without needing live tapeworms.

In one aspect, the compositions can be prepared from helminthic parasites that are obtained from preparatory animals or insects that are raised in a non-SPF (specific pathogen free) environment, such as a human specific pathogen free environment. The preparatory process can include isolating only the cysticercoids (e.g., not the eggs which are in the rat poop), and putting the cysticercoids in a buffer solution with antimicrobials and a preservative. However, the eggs can be obtained from rat poop, and optionally processed by artificial pH modulation as described.

In one aspect, the preparatory animal or preparatory insect is always in a “non-SPF environment” while having the helminthic parasite,

The manufacture process of the helminthic parasite preparation can be performed under the guidelines of good manufacturing, such as the good manufacturing process for supplements implemented by the FDA. The Final Rule establishes, at 21 C.F.R. Part 111, the minimum CGMPs required for manufacturing, packaging, labeling, or holding dietary supplements to ensure the quality of the dietary supplements. The Final Rule's sixteen subparts, described in detail in the 208-page Federal Register document, are driven by a few major themes that FDA reiterates throughout the preamble to the Final Rule: CGMPs are needed to ensure the quality of dietary supplements. Ensuring quality means that a company consistently and reliably manufactures what it intends and that it establishes process controls to prevent the dietary supplement from becoming adulterated.

•CGMPs require written procedures detailing the specifications a manufacturer establishes for its dietary supplements and the production processes needed to achieve those specifications. CGMPs require the creation of written documentation substantiating that these procedures are satisfied and the specifications are met.• The key steps towards ensuring the quality of dietary supplements are ensuring the identity and quality of components used to manufacture a dietary supplement, and utilizing in-process controls to ensure that the product specifications are met. These two requirements permit testing of only a subset of finished batches of product, rather than testing of each finished batch. CGMPs do not address the inherent safety of the ingredients used in dietary supplements, which is covered by other statutory provisions. Similarly, adverse event reporting requirements are not comprehensively encompassed within CGMPs. As such, there are non-trivial differences in the standards for pharmaceuticals versus dietary supplements. Housing requirements and carriers/delivery methods which are prohibited from current pharmaceutical manufacturing are allowed in the dietary supplement manufacturing process. Therefore, the method of manufacturing the helminthic parasite compositions can follow dietary supplement manufacturing protocols, and can be assessed with the FDA standards recited above. Thus, the method of manufacturing the helminthic parasite compositions can omit the requirements for pharmaceutical compositions.

In one embodiment, the invention can use activated cysticercoids. Cysticercoids are activated naturally when they pass from the stomach into the small bowel of an animal. This is also called excystation as they come out of the cyst. This is their transition phase from living in a beetle to living in a rat for H. diminuta. It is a common lab experiment to do it artificially by manipulating the pH of their environment and adding stomach/small bowel enzymes. The reason this may be helpful, is because only when they are activated and growing into adult tapeworms (in the small bowel) would they produce factors which affect the rat/human and modulate the immune system thereof or provide other therapeutic benefit. So it might be possible to activate them, and then use the dead activated ones (or an extract) in an enteric coated capsule to deliver the intended effect in the person without needing live tapeworms.

In one embodiment, the compositions and methods can use soluble extract of any part of the life cycle (e.g., adult, egg, or cysticercoid). These can be extracted with water, ethanol, or oil. The parasite in any part of the life cycle can also be lyophilised. Cysticercoids could be activated in the lab prior to extraction or administration. After activation they could be grown for a period of time. It could also be possible to mimic an attacking immune system so that they produce more immune modulation substances.

In some embodiments, some helminthic parasites may be specifically omitted or not used in the compositions or methods. The helminthic parasites to avoid or not use can include those listed in the applications and patents recited in the background or in the references that are incorporated herein by specific reference.

In one embodiment, the intermediate host or preparatory animal is an insect or other arthropod. The insect or arthropod can be selected from grasshoppers, crickets, earwigs, fleas, beetles, butterflies and moths, and even millipedes or centipedes as well as specifically T. molitor and T. confusum.

Generally, the compositions and methods can use any helminthic parasite. However, some parasites, as described below, can be preferred. Hymenolepis diminuta is the most preferred helminthic parasite.

Hymenolepis diminuta can be used in the present invention, and is known to colonize people occasionally, and when it does colonize people there are no/few untoward effects reported. Hymenolepis diminuta, also known as rat tapeworm, is a species of Hymenolepis tapeworm that causes hymenolepiasis. It has slightly bigger eggs and proglottids than H. nana and infects mammals using insects as intermediate hosts. The adult structure is 20 to 60 cm long and the mature proglottid is similar to that of H. nana, except it is larger.

The cycle begins as arthropods ingest the eggs. Arthropods are then able to act as the intermediate host. When ingested, the eggs develop into cysticercoids. Oncospheres hatch and then penetrate the intestinal wall. Rodents can become infected when they eat arthropods. Humans, especially children, can ingest the arthropods as well and therefore become infected via the same mechanism. Rodents, especially rats, are definitive hosts and natural reservoirs of H. diminuta. The intermediate hosts are the coprophilic arthropods (fleas, lepidoptera, and coleoptera). As the definitive host (rats) eats an infected arthropod, cysticercoids present in the body cavity transform into the adult worm. The resulting eggs are then passed through the stool. In recent findings, beetle-to-beetle transmission of H. diminuta can be seen via the feces. Additionally, more infections occur due to this mechanism of egg dispersal.

In a behavioral study of the beetle Tenebrio molitor with cysticercoids of the rat tapeworm H. diminuta, findings suggested that the parasite impairs a beetle's ability to conceal itself. The study followed a rat and a beetle infected with the parasite. Infected beetles were slower than the control group; however, they still maintained the same learning level. In the initial phase of infection, the beetle was in high stress. As time progressed, this did not worsen their ability to learn. Overall, the training experiment portrayed that infected beetles were unable to hide from the rat, illustrating the high impact the parasite had on its host, the beetle.

In one embodiment, the therapeutic methods of the present invention can include administering to a subject a substance that causes removal of the H. diminuta from the subject. Such removal can be by niclosamide treatment, which as been proved to be successful. Thus, the therapy can be terminated by such administration.

H. diminuta is one of the most common (perhaps the most common) parasites studies in the lab. It is known to colonize man with little to no side effects. Other helminthes are thought to help with immune dysfunction and autoimmune diseases. It is surprising that nobody has thought of using H. diminuta for these purposes in humans. It has also been folklore/known for years that tapeworm colonization is associated with being thin. It is surprising that nobody has suggested using H. diminuta for the purpose of losing weight.

The rat tapeworm, Hymenolepis diminuta, is a well-studied laboratory parasite. For the last fifty years it has been used to teach secondary and college students the life cycle of cestodes. Carolina Biological Supply currently ships Hymenolepis diminuta in both egg and cysticercoid state within the United States without restriction. Despite accessibility it has not been used in humans for the purpose of modulating immune dysfunction and/or metabolic function, which now are methods of the present invention.

Hymenolepis diminuta has a number of advantages over current helminthes being studied for the purpose of modulating the human immune system. Hymenolepis diminuta is easily and inexpensively kept in the lab. A suitable intermediate host, Tenebrio molitor, is already in the food supply at allowable levels set by the Food and Drug Administration (FDA). Thus, the tenebrio molitor can be used for preparing the compositions, used as the compositions, and in the therapies.

The Centers for Disease Control classifies Hymenolepis diminuta as a class I organism requiring the lowest level described for biosafety and does not require elaborate regulatory oversight or bio-containment protocols. Additionally, Hymenolepis diminuta is a nutrient feeder that attaches to the intestinal mucosa via a suckered scolex. The scolex of H. diminuta does not have an armed rostrellum and does not leave lesions or ulceration at the attachment site. As H. diminuta develops in intestinal lumen the established, but not persistent, infections grow rapidly requiring additional immune modulation prior to expulsion.

In one embodiment, the cysticercoids or live adult H. diminuta can be administered orally or rectally such as with a suppository or other administration medium. The administration medium can be liquid, gel, paste, or solid or particulate, which can be consumed or rectally administered.

A lab was set up that consists of two Norwegian hooded rats. Each six month old rat was infected with ten cysticercoids obtained from beetles, Tenbrio molitor, supplied by Carolina Biologic Supply. The tapeworms were allowed to mature to gravid organism as verified by microscopic ova detection of fecal samples. Feces containing Hymenolepis eggs were fed to Tenebrio molitor beetles supplied by Carolina Biologic, Ward's Scientific and mealworms obtained from local Petsmart raised to adult beetles. Hymenolepis Diminuta cysticercoids (HDCs) were harvested from the adult beetles starting at three weeks. These cysticercoids were used to self-inoculate to verify effect. Self-inoculation doses ranged from 5 to a complete beetle which we estimate to contain between 20-300 cysticercoids. HDCs were subjected to activation to verify viability. Selected cysts were removed from harvested doses and treated with a gastric proxy, then a trypsin/bile salt solution until active, motile scolexes were microscopically visualized excysted from oncospheres.

Harvested doses are then triple washed in a buffer solution consisting of Tyrode's solution. After the final wash cysts are placed in cryovials in buffer solution with antimicrobials and glucose (dextrose). The approximate shelf life of this product is six days.

Doses were given to a range of patients with conditions of Th1/Th2 dysregulation in a dose of 15 QD×2. The preferred re-enforcement schedule is this dose every three weeks. There is typically no or very minimal response to the first dose. The second dose typically sees an increase in immune modulation events (IME) including constipation, fever, night sweats and transient anxiety. The third dose very often presents with increased immune modulation events as well as reportable improvement in the Th1/Th2 dysregulation. Subsequent doses tend to have diminished or reduced IMS with the exception of further improvement of Th1/Th2 dysregulation symptoms. Doses should be continued every three weeks to maintain benefit. There are instances where Th1/Th2 dysregulation is too active and termination of the therapy can be achieved with a single dose of praziquantel at 25 mg/kg.

In one embodiment, Dipylidium caninum can be a preferred helminthic parasite, which is a common organism of cats and dogs, but known to colonize people occasionally. When it does colonize people there are no/few untoward effects reported. Also, the intermediate host is an insect (flea), which can be used for preparing the composition or as the composition. That is, the flea having the D. caninum can be consumed for the therapy or used in preparing a therapeutic composition. Other insect hosts can be used.

Dipylidium caninum, also called the cucumber tapeworm or the double-pore tapeworm, is a cyclophyllid cestode that infects organisms afflicted with fleas, including canids, felids, and pet-owners, especially children. Adult worms are about 18 inches long. Eggs (or “egg clusters” or “egg balls”) are passed in the host's feces and ingested by fleas, which are in turn ingested by another mammal after the tapeworm larvae partially develop. Examples of fleas that can spread D. caninum include Ctenocephalides canis and Ctenocephalides felis.

As in all members of family Dipylidiidae, proglottids of the adult have genital pores on both sides (hence the name double-pore tapeworm). Each side has a set of male and female reproductive organs. The scolex has a rostellum with four rows of hooks, along with the four suckers that all cyclophyllid cestodes have. Inside fleas, eggs hatch and form oncosphere larvae that move through the wall of the flea intestine into the body cavity where they become cysticercoid larvae, which are infective to mammal hosts. In children, infection causes diarrhea and restlessness. As with most tapeworm infections, the drugs of choice are niclosamide or praziquantel for removal. The best way to prevent human infection is to treat infected animals and to kill fleas. Although, D. Caninum is usually transferred via a flea, Trichodectes canis, the chewing louse of dogs, can also be the intermediate host for the tapeworm. D. caninum is a very common cat and dog parasite that is also known to colonise man with little/no untoward effects at low dose. It is also surprising that nobody has suggested using this organism for weight regulation or immune regulation or as a dietary supplement.

In one embodiment, one or more Diphyllobothrium species can be used in the compositions and methods of the present invention. Many species of Diphyllobothrium can colonise man. The species that is most known and discussed is D. latum. They have complicated life cycles that include 3 intermediate hosts. Usually one crustacean and two fish. When these colonise man there is usually little/no untoward effects reported at low dose.

Diphyllobothrium is a genus of tapeworm which can cause Diphyllobothriasis in humans through consumption of raw or undercooked fish. The principal species causing diphyllobothriosis is Diphyllobothrium latum, known as the broad or fish tapeworm, or broad fish tapeworm. D. latum is a pseudophyllid cestode that infects fish and mammals. D. latum is native to Scandinavia, western Russia, and the Baltics, though it is now also present in North America, especially the Pacific Northwest. In Far East Russia, D. klebanovskii, having Pacific salmon as its second intermediate host, was identified. Other members of the genus Diphyllobothrium include Diphyllobothrium dendriticum (the salmon tapeworm), which has a much larger range (the whole northern hemisphere), D. pacificum, D. cordatum, D. ursi, D. lanceolatum, D. dalliae, and D. yonagoensis, all of which infect humans only infrequently. In Japan, the most common species in human infection is D. nihonkaiense, which was only identified as a separate species from D. latum in 1986. It was indicated to be synonymous to D. klebanovskii from the molecular study

The adult worm is composed of three fairly distinct morphological segments: the scolex (head), the neck, and the lower body. Each side of the scolex has a slit-like groove, which is a bothrium (tentacle) for attachment to the intestine. The scolex attaches to the neck, or proliferative region. From the neck, grows many proglottid segments which contain the reproductive organs of the worm. D. latum is the longest tapeworm in humans, averaging ten meters long. Adults can shed up to a million eggs a day.

In adults, proglottids are wider than they are long (hence the name broad tapeworm). As in all pseudophyllid cestodes, the genital pores open midventrally.

Adult tapeworms may infect humans, canids, felines, bears, pinnipeds, and mustelids, though the accuracy of the records for some of the nonhuman species is disputed. Immature eggs are passed in feces of the mammal host (the definitive host, where the worms reproduce). After ingestion by a suitable freshwater crustacean such as a copepod (the first intermediate host), the coracidia develop into procercoid larvae. Following ingestion of the copepod by a suitable second intermediate host, typically a minnow or other small freshwater fish, the procercoid larvae are released from the crustacean and migrate into the fish's flesh where they develop into a plerocercoid larvae (sparganum). The plerocercoid larvae are the infective stage for the definitive host (including humans).

Because humans do not generally eat undercooked minnows and similar small freshwater fish, these do not represent an important source of infection. Nevertheless, these small second intermediate hosts can be eaten by larger predator species, for example, trout, perch, and walleyed pike. In this case, the sparganum can migrate to the musculature of the larger predator fish and mammals can acquire the disease by eating these later intermediate infected host fish raw or undercooked. After ingestion of the infected fish, the plerocercoids develop into immature adults and then into mature adult tapeworms which will reside in the small intestine. The adults attach to the intestinal mucosa by means of the two bilateral grooves (bothria) of their scolex. The adults can reach more than 10 m (up to 30 ft) in length in some species such as D. latum, with more than 3,000 proglottids. One or several of the tape-like proglottid segments (hence the name tape-worm) regularly detach from the main body of the worm and release immature eggs in fresh water to start the cycle over again. Immature eggs are discharged from the proglottids (up to 1,000,000 eggs per day per worm) and are passed in the feces. The incubation period in humans, after which eggs begin to appear in the feces is typically 4-6 weeks, but can vary from as short as 2 weeks to as long as 2 years. The tapeworm can live up to 20 years.

In one embodiment, the therapeutic methods can include administering a substance that causes removal of this helminthic parasite from a subject. A single dose of Praziquantel, 5-10 mg/kg PO once for both adults and children can be used. An alternative treatment is Niclosamide, 2 g PO once for adults or 50 mg/kg PO once. Another interesting potential diagnostic tool and treatment is the contrast medium, Gastrografin, introduced into the duodenum, which allows both visualization of the parasite, and has also been shown to cause detachment and passing of the whole worm. These substances may be useful for removal of other helminthic parasites used in the therapeutic methods described herein.

Diphyllobothrium species have been reported to cause weight loss but have not been used for this purpose or for immune regulation. Therefore, now, Diphyllobothrium species can be administered with the specific intent for these purposes, such as to subjects in need thereof. As such, the subject can be specifically selected and provided the Diphyllobothrium species, and the subject can self-administer the Diphyllobothrium species on purpose. The same can occur for other helminthic parasites described herein.

In one embodiment, Hymenolepis nana can be used in the compositions and methods of the present invention. There is good evidence that there are 2 species of H. nana. One colonizes man and the other colonizes rats. There is some confusion in this matter in the literature however.

Dwarf tapeworm (Hymenolepis nana, previously known as Vampirolepis nana, Hymenolepis fraterna, and Taenia nana) is a cosmopolitan species though most common in temperate zones, and is one of the most common cestodes of humans, especially children.

As its name implies (Greek: nanos—dwarf), it is a small species, seldom exceeding 40 mm long and 1 mm wide. The scolex bears a retractable rostellum armed with a single circle of 20 to 30 hooks. The scolex also has four suckers, or a tetrad. The neck is long and slender, and the segments are wider than long. Genital pores are unilateral, and each mature segment contains three testes. After apolysis gravid segments disintegrate, releasing eggs, which measure 30 μm to 47 μm in diameter. The oncosphere is covered with a thin, hyaline, outer membrane and an inner, thick membrane with polar thickenings that bear several filaments. The heavy embryophores that give taeniid eggs their characteristic striated appearance are lacking in this and the other families of tapeworms infecting humans.

Infection is acquired most commonly from eggs in the feces of another infected individual, which are transferred in food, by contamination. Eggs hatch in the duodenum, releasing oncospheres, which penetrate the mucosa and come to lie in lymph channels of the villi. Oncospheres develops into a cysticercoid which has a tail and a well formed scolex. It is made of longitudinal fibers and is spade shaped with the rest of the worm still inside the cyst. In five to six days cysticercoids emerge into the lumen of the small intestine, where they attach and mature.

The direct life cycle is doubtless a recent modification of the ancestral two-host life cycle, found in other species of hymenolepidids, because cysticercoids of H. nana can still develop normally within larval fleas and beetles. One reason for facultative nature of the life cycle is that H. nana cysticercoids can develop at higher temperatures than can those of the other hymenolepidids. Direct contaminative infection by eggs is probably the most common route in human cases, but accidental ingestion of an infected grain beetle or flea cannot be ruled out. The direct infectiousness of the eggs frees the parasite from its former dependence upon an insect intermediate host, making rapid infection and person-to-person spread possible. The short life span and rapid course of development also facilitate the spread and ready availability of this worm.

Hymenolepis nana, like all tapeworms, contains both male and female reproductive structures in each proglottid. This means that the dwarf tapeworm like other tapeworms is hermaphroditic. Each segment contains three testes and a single ovary. When a proglottid becomes old and unable to absorb any more nutrition, it is released and is passed through the host's digestive tract. This gravid proglottid contains the fertilized eggs, which are sometimes expelled with the feces.

In one embodiment, the therapeutic methods can include removal of this parasite from the subject. The two drugs that have been described for the treatment of hymenolepiasis are praziquantel and niclosamide. Praziquantel, which is parasiticidal in a single dose for all the stages of the parasite, is the drug of choice because it acts very rapidly against H. nana. Although structurally unrelated to other anthelminthics, it kills both adult worms and larvae. In vitro the drug produces vacuolization and disruption of the tegument in the neck of the worms, but not in more posterior portions of the strobila. Praziquantel is well absorbed while taken orally, it undergoes first-pass metabolism and 80% of the dose is excreted as metabolites in urine within 24 hours. Preventing fecal contamination of food and water in institutions and crowded areas is of primary importance. General sanitation and rodent and insect control (especially control of fleas and grain insects) are also essential for prevention of H. nana infection.

In on embodiment, the helminthic parasite can be a tapeworm (cestode). All of the parasitic helminths currently being used or researched by others (including Weinstock) are roundworms (nematodes), which as substantially different from cestodes. All parasites tend to get lumped together, but these are very different kinds of animals. This distinction may also make a difference in the therapy. Previously, tapeworms have not been used in the methods of the present invention. Surprisingly, it has been found that indeed tapeworms can be used in the methods of the present invention.

In one embodiment, tapeworms that belong to the class Cestoda of the phylum Platyhelminthes can be used as the helminthic parasites of the present invention in the compositions and methods. The specific tapeworms can include: Hymenolepis diminuta, Hymenolepis nana, Diphyllobothrium latum, Diphyllobothrium pacificum, Diphyllobothrium dendriticum, Diphyllobothrium nihonkaiense, Diphyllobothrium cordacum, Diphyllobothrium ursi, Diphyllobothrium lanceolatum, Diphyllobothrium dalliae, and Diphyllobothrium yonagoensis.

In one embodiment, the methods of manufacturing the helminthic parasite composition specifically excludes use of a human specific pathogen free environment. That is, the environment can be general and not characterized or prepared in any way to be pathogen free or free of human pathogens. In one example, the environment used in the method, such as for raising the preparatory animal or preparatory insect can have one or more known human pathogens. The one or more human pathogens that are included in the environment of the preparatory animal or preparatory insect can include one or more of the following: Viruses, Rabbit Hemorrhagic Disease Virus, Myxomatosis, Bacteria, Mycoplasma and Fungi, Bordetella bronchiseptica, CAR Bacillus, Clostridium piliforme, Helicobacter bilis Helicobacter hepaticus Helicobacter spp, Pasteurella multocida Pasteurella pneumotropica, Salmonella spp, Toxoplasma spp, Treponema cuniculi, Parasites, Ectoparasites, Endoparasites, Enteric Protozoan, Encephalitozoon cuniculi, Ectromelia Virus, Hantaan Virus, Lactic Dehydrogenase Elevating Virus, Lymphocytic Choriomeningitis Virus (LCM), Minute Virus of Mice, Adenovirus type 1, Mouse Adenovirus type 2 (K87), Mouse Cytomegalovirus, Mouse Hepatitis Virus, Mouse Parvovirus, Mouse Polyoma Virus, Mouse Rotavirus (EDIM), Mouse Thymic Virus, Murine Norovirus, Pneumonia Virus of Mice, Respiratory Enteric Virus III (REO 3), Sendai Virus, Theiler's Murine Encephalomyelitis Virus (TMEV, GD7), Bacteria, Mycoplasma and Fungi, Porcine reproductive and respiratory syndrome virus, Actinobacillus pleuropneumonia, Mycoplasma hypopneumoniae, Sucking Louse, Burrowing mite, Porcine Pseudorabies Virus, Brucella abortus, Avian Adenovirus Group I, Avian Adenovirus Group II (HEV), Avian Adenovirus Group III (EDS), Avian Encephalomyelitis, Avian Influenza (Type A), Avian Nephritis Virus, Avian Paramyxovirus Type 2, Avian Reovirus S 1133, Avian Rhinotracheitis Virus; Avian Rotavirus; Avian Tuberculosis M. avium; Chicken Anemia Virus; Endogenous GS Antigen; Fowl Pox; Hemophilus paragallinarum Serovars A, B, C; Infectious Bronchitis—Ark; Infectious Bronchitis—Conn; Infectious Bronchitis—JMK; Infectious Bronchitis—Mass; Infectious Bursal Disease Type 1; Infectious Bursal Disease Type 2; Infectious Laryngotracheitis; Lymphoid Leukosis A, B; Avian Lymphoid Leukosis Virus; Lymphoid Leukosis Viruses A, B, C, D, E, J; Marek's Disease (Serotypes 1, 2, 3); Mycoplasma gallisepticum; Mycoplasma synoviae; Newcastle Disease LaSota; Reticuloendotheliosis Virus; Salmonella pullorum-gallinarum; Salmonella species; staph aureus, and others. Some specific examples can include: viral pathogens, such as Adenovirus, Coxsackievirus, Hepatitis (A, B, C) virus, Herpes simplex virus, Cytomegalovirus, Influenza virus, Parainfluenza virus; bacterial pathogens, such as Campylobacter jejuni, Clostridium botulinum, Clostridium difficile, Clostridium perfringens, Clostridium tetani, Enterococcus faecalis, Enterococcus faecium, E. coli, Haemophilus influenzae, Pseudomonas aerugenosa, Staphylococcus aureus, Staphylococcus epidermidis, Streptococcus pneumoniae, Streptococcus pyogenes; and fungal pathogens, such as Aspergillus species, Candida species, Cryptococcus neoformans, Histoplasma capsulatum, Pneumocystis carinii, Stachybotrys chartarum (black mold). Additional pathogens can include: Bacillus altitudinis, Bacillus pumilus, Lysnibacillus boronitolerans, Bacillus kochii, and Bacillus safensis.

For example, there likely is fungi in the beetles as the intermediate host. Some of these bacteria are common in the environment as well and likely to be present in conventional housing. Most of the viruses and some of the bacteria could be from human contact with the beetles and their cages in the conventional housing.

Specific Pathogen Free (“SPF”) is a term used for laboratory animals that are guaranteed free of particular pathogens. It is always accompanied by a list of the absent pathogens.

Use of SPF animals ensures that specified diseases do not interfere with an experiment. For example, absence of respiratory pathogens such as influenza is desirable when investigating a drug's effect on lung function. In some embodiments, the present invention can use a SPF environment. In other methods, a SPF environment is not used.

When by accident some infection does occur, the population is said to have minimal disease status. minimal disease status is not specific pathogen free as there is a pathogen that can cause infection.

Rather, the methods of manufacturing the helminthic parasite composition or preparing the helminthic parasite can be in a conventional environment or conventional housing. Conventional environment or housing is not specific pathogen free.

Animals can be conventionally housed in open caging feed water bedding and caging components are not autoclaved prior to use. Rodents or insects will have their cages, water, and feed changed on a weekly basis more often if the situation requires it, wet cages or feed etc. Caging components and water bottles and stoppers are sanitized prior to use.

In one embodiment, the present invention can using helminthic parasite tape worms that do not typically form persistent infections in man and using immune suppressants to humanize the worms. That is, the helminthic parasite can be administered to a human along with, before, or after administration of an immune suppressant. This immune suppressant can enable the helminthic parasite to grow in a human subject even though the helminthic parasite is not a specific human parasite.

In one embodiment, the present invention can include the use of an intermediate host (e.g. beetle) to sterilize the infective stage that has been kept in an environment known to have specific human pathogens. That is, the cysticercoids can be within a beetle host while the beetle lives in a non-SPF environment, and then the cysticercoids can be obtained from the beetle. For example, the cysticercoids can be obtained from the sterile inner part of the beetle abdomen.

In one embodiment, the present invention can use the helminthic parasite compositions for the purpose of maturing the immune system of a subject, such as described herein.

In one embodiment, the present invention can use the helminthic parasite compositions for the purpose of improving the immune system of a subject, such as described herein.

In one embodiment, the present invention can use the helminthic parasite compositions for the purpose of normalizing the bacterial populations in the intestinal tract of a subject.

In one embodiment, the present invention can use the helminthic parasite compositions for the purpose of balancing metabolic function of a subject.

In one embodiment, the present invention can use the helminthic parasite compositions for the purpose of to introduce bacteriostatic molecules into the intestinal lumen for the purpose of remodeling the microbiome of a subject.

The theory is this, without being bound thereto that as the human immune system evolved it was constantly battling worms that lived in the intestinal tract. At some point, the immune system conceded the worms would be there no matter what the immune system did and simply evolved around their presence (scientifically called mutualistic symbiosis). When this happens, the worms and the immune system created a partnership. The worms were allowed to feed and the immune system used some chemicals the worms secreted to function normally.

This process is a type of “enzyme sharing” and it occurs with the bacteria (called commensal or mutalistic bacteria) in the intestinal tract all the time. The consequence is that without the worms the immune system does not function properly, and in your case starts to attack self or harmless substances that it should be ignoring. What our method does is re-introduces the safer worms so that immune function can be optimized.

A partial mechanism for how this is accomplished is that certain worms secrete a chemical that makes attacking immune cells into regulatory immune cells (the worms have to turn down the immune response to survive in the harsh gastrointestinal environment). These regulatory immune cells are called regulatory T cell, B cells, macrophages and dendritic cell=good guys. When the worms create enough of these regulatory immune cells they circulate throughout your body and make more regulatory immune cells. The overall effect of this is to modulate a balance between Th1 and Th2 function. The expansion and circulation of regulatory immune cells is how we can get effect inside and outside of the intestinal tract. This can be beneficial in the present therapeutic methods.

An interesting and unusual finding is that, through the process of evolution, the immune system has worked out how to optimize its function when it has enough regulatory immune cells. So the immune system in the presence of some intestinal worms ignores the good or harmless stuff (dander, pollen) while still responding vigorously to invaders that can hurt you (like pathogenic bacteria, fungus or viruses). We have interesting and unexpectedly observed clinically that individuals with regular viral outbreaks are better controlled in the context of hosting worms. Thus, the therapeutic methods can include method of inhibiting allergies, while increasing protection against virus or other germ pathogens.

As such, the helminthic parasites can be used in therapies for allergic conditions, autoimmunity, and any disease and or conditions that results from an imbalance of Th1/Th2 function. That is, the helminthic parasites can be used to balance Th1/Th2 function. Additionally, the worms will change bacterial populations that live in the intestinal tract. These bacterial populations have an essential role in maturing the immune system thus the therapy expands to their use in maturing the human immune system. That is, deliberate infections at specific ages for the purposes of assisting the immune system develop optimally.

Finally, tapeworms have a well-known role in weight loss, but are usually not affirmatively and specifically administered for weight loss, but now the present invention includes administration for specific weight loss.

Now, the helminthic parasites can be used to treat individuals who have Inflammatory Bowel disease or intestinal autoimmunity often times have a very difficult time maintaining or gaining weight. Clinically we have made the interesting and unusual finding that tapeworms can help certain individuals gain weight. That is, the therapy is useful in reversing metabolic challenges of different natures.

In one embodiment, the helminthic parasites can be prepared and used as described. A lab was set up that consists of two Norwegian hooded rats. Each six-month-old rat was infected with ten cysticercoids obtained from beetles, Tenbrio molitor, supplied by Carolina Biologic Supply. The tapeworms were allowed to mature to gravid organism as verified by microscopic ova detection of fecal samples. Feces containing Hymenolepis eggs were fed to Tenebrio molitor beetles supplied by Carolina Biologic, Ward's Scientific and mealworms obtained from local Petsmart raised to adult beetles. Hymenolepis diminuta cysticercoids (HDCs) were harvested from the adult beetles starting at three weeks. These cysticercoids were used to self-inoculate to verify effect. Self-inoculation doses ranged from 5 to a complete beetle which we estimate to contain between 20-300 cysticercoids. HDCs were subjected to activation to verify viability. Selected cysts, approximately 20-50, were removed from harvested doses and treated with a gastric proxy consisting of HCl acid and Pepsin for ten minutes at 37 degrees C., then a trypsin/bile salt solution until active (typically 10-20 min), motile scolexes are then microscopically apparent excysted from oncospheres.

Harvested doses are then triple washed in a buffer solution consisting of Tyrode's solution. After the final wash cysts are placed in cryovials in 0.65% sodium chloride/purified water (USP) buffer solution containing phenylcarbinol, benzalkonium chloride and 0.2 g/ml glucose (dextrose). The approximate shelf life of this product is five days at room temperature and six days refrigerated. Doses were given to a range of patients with conditions of Th1/Th2 dysregulation in a dose of 15 QD×2. The split dosing is used to avoid the crowding effect. The preferred re-enforcement schedule is this dose every three weeks.

It may be possible for there to be instances where Th1/Th2 dysregulation is too active to tolerate the six to nine week ramp up and it becomes necessary to abrogate the therapy. This is accomplished by a single dose of praziquantel at 25 mg/kg.

The present invention can include various dosing regimens. There can be a range of dose preparations: A 21 gauge needle can be filled with 0.5 ml of sterile saline (USP) and injected into the abdominal cavity and the fluid aspirated out and into a sterile ampule containing buffer solution. This dose can then be mixed with appropriate supplement carrier e.g. chocolate milk.

A smaller species of beetle, Tribolium confusum or more commonly known as the rice flour beetle, can be used to prepare doses in the 0-20 range. This beetle can live without water inside a gelatin capsule for months. Such preparation will entail placing a complete beetle in a gelatin capsule with 0.1 gram rice flour.

The entire wheat beetle can be placed in 96% ethanol and exposed to two minutes of UV light at approximately 260 nm. This product can then ingested in complete form, placed in a gelatin capsule or can be dipped in chocolate or yogurt.

A different suitable intermediate host can be infected that is already eaten in complete form e.g. grasshopper or ant.

A potentially superior method of dosing may be in establishing persistent infections using any of the previously described dosing techniques in the context of deliberate immune suppression in the patient.

In one embodiment, the present invention can include methods for establishing persistent infections. For example, one budesonide 3 mg extended release (Entocort) is taken every 12 hours starting 12 hours before inoculation with 60 cysticercoids. Budesonide at 3 mg bid is taken for five days post inoculation. Additionally, omeprazole 20 mg (Prilosec OTC) and loratadine 10 mg are taken daily for entire period of infection. Testing for presence of adult patent H. diminuta is performed starting at approximately 14 days post inoculation to confirm infection.

In one embodiment, the compositions having the helminthic parasite can be dietary supplements or used as dietary supplements, but not pharmaceuticals. The Dietary Supplement and Health Education Act 1994 defines supplement thusly: “A dietary supplement is a product taken by mouth that contains a “dietary ingredient” intended to supplement the diet. The “dietary ingredients” in these products may include: vitamins, minerals, herbs or other botanicals, amino acids, and substances such as enzymes, organ tissues, glandulars, and metabolites. Dietary supplements can also be extracts or concentrates, and may be found in many forms such as tablets, capsules, softgels, gelcaps, liquids, or powders. They can also be in other forms, such as a bar, but if they are, information on their label must not represent the product as a conventional food or a sole item of a meal or diet. Whatever their form may be, DSHEA places dietary supplements in a special category under the general umbrella of “foods,” not drugs, and requires that every supplement be labeled a dietary supplement.”

Secondly, there are different standards for Supplement vs. Pharmaceutical in Current Good Manufacturing Processing (GMP)—specifically in the bioburden allowances.

For example, the standards allowed for an acceptable pharmaceutical carrier in aqueous preparations for oral use are: Total Aerobic Microbial Count (cfu/ml) 10²; Total combined Yeasts/Molds Count (cfu/ml) 10¹; Absence of Escherichia Coli (1 mL)

On the other hand, for supplement carrier: Total Aerobic Microbial Count NMT* 10³ cfu; and Total Yeast/Molds Count NMT 10² cfu; and Absence of Escherichia Coli (10 mL). (*Not more than)

In one embodiment, the methods can include cleaning the preparatory beetle on the outside, and then sticking a needle into the appropriate place in the abdomen, and pull out the cysticercoids. This can produce cysticercoids in a sterile fashion.

In one embodiment, the helminthic compositions can be used in methods of reducing bacteria in the gut. By administration of the helminthic compositions, bacteria can be removed from the gut of a subject, such as a human.

In one embodiment, the helminthic compositions can be used in methods of treating one or more of the following: allergies, Crohn's disease, ulcerative colitis, asthma, G.I. complications, celiac esophagitis, E-acidophilic gastritis, or the like.

In one embodiment, the therapeutic methods include providing helminthic parasite tapeworm protein to the subject. That is, the therapy provides a subjects' immune system with worm protein, and then the immune system will start to have this TH2 allergic response, which will then balance the TH1. However, while the tapeworm proteins can provide the therapy, the effect can improve as you provide more protein, then more effect as you move up the life cycle, such as from the eggs, then more effect from the cysticercoid, and you get even more effect from a transient infection with an adult tapeworm, and then you get even more effect if you actually have a persistent adult worm living inside the subject. As such, humanizing the tapeworm by administration with immune system inhibitors can be advantageous.

In one embodiment, the methods of manufacture can include obtaining the beetles already having the cysticercoids. Then prepare the doses. This can include obtaining alive or dead beetle having the cysticercoids.

In one embodiment, the method of manufacture includes the beetles not being raised in a specific germ free or pathogen free environment. That is, the environment is conventional or can have a human pathogen.

In one embodiment, the cysticercoids may be obtained sterile directly from the beetle. That is, the cysticercoids are not isolated or purified in any matter. Also, obtaining sterile cysticercoids can avoid having to clean them up, because where they live inside the beetle is already sterile—the abdominal cavity of the beetles is sterile. Then the method can include aspirating the cysticercoids directly from the abdominal cavity. Optionally, this method can include cleaning the outside with a sterilizing solution, and then aspirating out of the sterile part of the abdomen having the cysticercoid.

In one embodiment, the compositions can include helminthic parasites, portions thereof, attenuated organism, extracts thereof, substances thereof, which can be from any part of the lifecycle thereof. The extracts can be soluble extracts that are soluble in water, ethanol, oil, or the like. The attenuated organism can be injured or partially dead, but still alive). As such, the composition could have the live worm, partially attenuated worm, it could have the eggs in it or it could have the cysticercoids in it. Soluble extracts can be obtained by grind the parasite up, and then whatever floats out in the extract solution can be the extract. For example, take the eggs and grind them up, and put them in an extract or take an extract of the worm itself. In some instances, cleaning the cysticercoids or live worm can be thorough, with a solution, or so rigorously that you're going to wind up with a partially alive worm that's still alive but it can't function as a fully healthy worm, which is an attenuated tapeworm.

In one embodiment, the composition can include cysticercoids that are alive, and a source of energy. The energy source can be glucose, galactose and/or amino acids.

In one embodiment, one method includes administering a beetle having the cysticercoid of the tapeworm.

In one embodiment, a method can include providing beetles having the cysticercoids for consumption with instructions for use. A therapeutic kit can provide the subject with the beetle and instructions on how to use it. The kit can include enough beetles to provide a therapeutically effective amount of cysticercoids in a beetle form. The therapeutically effective amount can be sufficient for the regimes described.

In one embodiment, the composition can be provided by: obtaining the beetle having the cysticercoids, squish out the contents of the abdomen of the beetle, and mixing the contents with chocolate or some other food source.

The regimen can vary. In one aspect, the dosing regimen can include administering 2 doses of 15 cysticercoids every 3 weeks. In one aspect, the dose can be once a day for two days every three weeks. The dose can be from 10 to 100, or from 11 to 50, or from 12 to 25, or from 13 to 20 or about 15 cysticercoids.

In one example, the dosing can be once a day or twice a day, the subject takes 15 cysticercoids, and then wait until the next day and take 15 more, and then wait 3 weeks and take 15 more and then the next day take 15 more. The amount can deviate from 15, such as plus or minus 5 or 10 or 15 or 20 or 30, or the like. The amount can be a therapeutically effective amount.

In one option, the dosing can be administering 10 cysticercoids in the morning and 10 in the evening.

In one embodiment, the dosing can include 6-8 cysticercoids in the morning and 6-8 cysticercoids at night for two days in a row. The dosing can be repeated every week, every 2 weeks, every 3 weeks, every 4 weeks, every 5 weeks, once a month, or the like.

In one embodiment, the dosing can be performed along with an immune system suppressant in order to transform the transient infection into a longer ore more persistent infection. A subject can turn these infections into persistent infections by suppressing the immune system. In one example, the immune system suppressant Entocort can be used. Also, budesonide, which is a time-released steroid that is a capsule, can be used orally.

Suppressing the immune system to allow for a larger number of tape worms to allow a larger number of tape worms to survive in the gut to get these methods of weight loss or immune modulation.

In one embodiment, a composition can include: a therapeutically effective amount of a tapeworm helminthic parasite preparation. The tapeworm helminthic parasite may be obtained from an in vitro method, such as described herein. The tapeworm helminthic parasite can include the cysticercoids. The composition can also include a carrier. In one aspect, the carrier is a supplement acceptable carrier and not a pharmaceutically acceptable carrier. On one aspect, the helminthic parasite can include whole helminthic parasites, portions thereof, attenuated organism, eggs, cysticercoids, and extracts thereof, substances thereof, which can be from any part of the lifecycle thereof. In one aspect, the composition can include beetle, portion thereof, extracts thereof, and substances thereof having cysticercoids. In one aspect, the helminthic parasite is selected from: Hymenolepis diminuta, Hymenolepis nana, Diphyllobothrium latum, Diphyllobothrium pacificum, Diphyllobothrium dendriticum, Diphyllobothrium nihonkaiense, Diphyllobothrium cordacum, Diphyllobothrium ursi, Diphyllobothrium lanceolatum, Diphyllobothrium dalliae, and Diphyllobothrium yonagoensis. In one aspect, the helminthic parasite is Hymenolepis diminuta. In one aspect, the helminthic parasite is not those listed in the references that are incorporated herein by specific reference. In one aspect, the composition is a dietary supplement. In one aspect, the composition is not a pharmaceutical.

In one embodiment, a method of manufacturing a helminthic parasite composition can include: obtaining the helminthic parasite preparation; and combining the helminthic parasite preparation with a carrier. The helminthic parasite preparation can be obtained from the in vitro method described herein. The preparation from the in vitro method can then be combined with a carrier that can maintain the cysticercoids.

In one embodiment, a method of producing a supplement composition comprising a helminthic parasite preparation can include: (1) raising a preparatory animal in conventional housing; and (2) mixing the host preparatory animal and or parasite isolate obtained from step (1) with a carrier, such as supplement acceptable carrier.

In one embodiment, a method of producing a supplement composition comprising a helminthic parasite preparation can include: (1) raising a preparatory insect in conventional housing; and (2) mixing the host preparatory insect and or parasite isolate obtained from step (1) with a supplement acceptable carrier.

In one embodiment, a method for producing a pharmaceutical or nutraceutical composition comprising rat-specific helminthic parasite in a medicinal, food, nutritional, or other comestible preparation, can include the steps of: (1) raising a preparatory animal in an environment that is not specific human pathogen-free; (2) isolating a helminthic parasite from said preparatory animal to form a helminthic parasite isolate; and (3) mixing the parasite isolate from step (2) with a carrier.

In one embodiment, a method for producing a pharmaceutical or nutraceutical composition comprising a helminthic parasite preparation, can include the steps of: (1) raising a preparatory insect; (2) isolating a helminthic parasite from said preparatory insect to form a helminthic parasite isolate; and (3) mixing the parasite isolate from step (2) with a carrier, such as a carrier that is not pharmaceutically acceptable.

In one embodiment, a method for producing a pharmaceutical or nutraceutical composition comprising a helminthic parasite preparation, can include the steps of: (1) raising a preparatory insect; (2) obtaining excrement from said preparatory insect; (3) isolating the helminthic parasite from the excrement; and (4) mixing the parasite isolate from step (3) with a carrier.

In one embodiment, a therapeutic method can include: providing the composition having the helminthic parasite preparation, such as prepared by the in vitro method; and administering the composition to a subject. In one aspect, the therapeutic method is for treating obesity. In one aspect, the therapeutic method is for treating the immune system. In one aspect, the therapeutic method is for treating a disease of the intestines. In one aspect, the therapeutic method is for modulating the Th1/Th2 response. In one aspect, the therapeutic method is for promoting weight loss. In one aspect, the therapeutic method is for maturing the immune system. In one aspect, the therapeutic method is for normalizing bacterial populations in the intestinal tract. In one aspect, the therapeutic method is for balancing metabolic function. In one aspect, the therapeutic method is for introducing bacteriostatic substances into the intestinal lumen for the purpose of remodeling the biome.

In one embodiment, a method of manufacturing a helminthic parasite composition can include: obtaining a tapeworm helminthic parasite preparation; and combining the helminthic parasite preparation with a carrier. The tapeworm helminthic parasite preparation can be obtained from the in vitro method, where the parasite is then combined with an oral carrier for oral consumption.

In one embodiment, a method of producing a composition comprising a tapeworm helminthic parasite preparation can include: (1) raising a preparatory animal; and (2) mixing the host preparatory animal and or tapeworm parasite isolate obtained from step (1) with a carrier.

In one embodiment, a method of producing a composition comprising a tapeworm helminthic parasite preparation can include: (1) raising a preparatory insect; and (2) mixing the host preparatory insect and or tapeworm parasite isolate obtained from step (1) with a carrier.

In one embodiment, a method for producing a composition comprising rat-specific helminthic parasite in a medicinal, food, nutritional, or other comestible preparation, can include the steps of: (1) raising a preparatory animal; (2) isolating a tapeworm helminthic parasite from said preparatory animal to form a helminthic parasite isolate; and (3) mixing the parasite isolate from step (2) with a carrier.

In one embodiment, a method for producing a composition comprising a tapeworm helminthic parasite preparation, can include the steps of: (1) raising a preparatory insect; (2) isolating a tapeworm helminthic parasite from said preparatory insect to form a helminthic parasite isolate; and (3) mixing the parasite isolate from step (2) with a carrier, such as a carrier that is pharmaceutically or nutraceutically acceptable.

In one embodiment, a method for producing a composition comprising a tapeworm helminthic parasite preparation, can include the steps of: (1) raising a preparatory insect; (2) obtaining excrement from said preparatory insect; (3) isolating the tapeworm helminthic parasite from the excrement; and (4) mixing the parasite isolate from step (3) with a carrier.

In any of the embodiments of the methods, such methods can be modified so that cysticercoids are obtained from an in vitro cell culture having been incubated in cell culture media. Then, the cysticercoids can be introduced into a consumable media, which can be a storage and shippable media that maintains the live cysticercoids. The live cysticercoids are capable of growing into worms inside a human gut.

In one embodiment, a method of restoring natural fauna to a gastrointestinal tract can include: obtaining a tapeworm helminthic parasite preparation composition, such as by the in vitro method; and administering the tapeworm helminthic parasite preparation to a subject in need thereof. In one aspect, the subject is a human having a fauna imbalance in their gastrointestinal tract.

### Shipping Solution:

In one embodiment, the present invention can include a shipping solution that has a cysticercoid. A shipping solution can contain essential nutrients for the cysticercoid form of the parasite to survive and also be safe for human consumption. For Hymenolepis diminuta cysticercoids (HDC) a solution containing amino acids, salts, organic acids, sugars, and antibiotic and antifungal agents can be used as a shipping solution. The shipping solution can also be human consumable. An example shipping solution containing these ingredients is as follows:

| | mg Per ml |
|---|---|
| H2O | 1000 |
| Alanine | 1.05 |
| Arginine | 2.30 |
| Aspartic acid | 1.92 |
| Cystine | 0.72 |
| Glutamic acid | 2.45 |
| Glycine | 1.80 |
| Histidine | 1.31 |
| Isoleucine | 1.79 |
| Leucine | 3.07 |
| Lysine | 2.27 |
| Methionine | 0.50 |
| Phenylalanine | 1.35 |
| Proline | 2.10 |
| Serine | 1.29 |
| Threonine | 1.50 |
| Tryptophan | 0.60 |
| Tyrosine | 1.29 |
| Valine | 1.95 |
| Glutamine | 0.21 |
| NaCl | 8.00 |
| KCl | 0.20 |
| CaCl2 | 0.20 |
| MgCl2 | 0.10 |
| NaH2PO4 | 0.05 |
| Glucose | 1.00 |
| NaHCO3 | 1.00 |
| Citric acid | 0.02 |
| Succinic acid | 0.01 |
| alpha ketoglutaric acid | 3.70 |
| Streptomycin sulphate | 1.50 |
| Trehalose | 7.00 |
| Malic acid | 0.30 |
| Fumaric acid | 0.30 |
| Yeast extract | 0.20 |
| Amphotericin B | 0.25 |
| HCl | 1.00 |

The shipping solution can be prepared by standard mixing and solution preparation protocols.

In one embodiment, a method of preparing a consumable shipping solution can be performed that results in a composition that maintains the live cysticercoids and can be ingested by a human. The following ingredients can be introduced into a sterilized screw top Duran flask that is protected from light, and which can be stirred with magnetic stirring, such ingredients include: Amphotericin B 250 mg/L dissolved in deionized water, and Nutricia Complete Amino Acid Mix 30 g/L. The amino acid mix contains per 100 g the following: Alanine at 3.5 g; Arginine at 7.66; Aspartic acid at 6.39 g; Cystine at 2.4 g; Glutamic acid at 8.17 g; Glycine at 6.0 g; Histidine at 4.38 g; Isoleucine at 5.95 g; Leucine at 10.22 g; Lysine at 7.55 g; Methionine at 1.65 g; Phenylalanine at 4.5 g; Proline at 7.0 g; Serine at 4.29 g; Threonine at 5.0 g; Tryptophan at 2.0 g; Tyrosine at 4.3 g; Valine at 6.5 g; and Glutamine at 0.7 g. Then, the composition is added with: NaCl 8.0 g/L, KCl, 0.2 g/L, CaCl₂ 0.2 g/L, MgCl₂ 0.1 g/L, NaH₂PO₄ 0.05 g/L, Glucose 1.0 g/L. NaHCO₃ 1.0 g/L, Streptomycin sulphate 1.5 g/L, alpha ketoglutaric acid 3.65 g/L, Trehalose 7.0 g/L, Citric acid 0.02 g/L, Fumaric acid 0.3 mg/L, Malic 0.3 mg/L, Succinic acid, 0.01 g/L, and Yeast extract 0.2 g/L. The pH is adjusted to 3.0 with HCl_{(aq)} 3M. The composition is then mixed, such as for 30 minutes or longer. Into a sterilized screw top Duran flask, filter the solution through Whatman Not filter paper. The composition can then be stored at 4° C. The composition is filtered through 0.2 μm membrane filter immediately before being combined with cysticercoids. The final composition having the HDC can include 1 mL of fluid for up to 180 HDC. While the composition can be used with Hymenolepis diminuta cysticercoids (HDC), it can also be used for other cysticercoids.

In one embodiment, the cysticercoids included in the composition can be obtained from in vitro cell culture methods.

### In Vitro Production:

In one embodiment, a parasite preparation can be manufactured using an in vitro protocol. It is useful to develop parasites for human use in vitro. This can greatly increase the production volume and ease of production. This could be any or all portions of the lifecycle including the portion (e.g., Hymenolepis diminuta cysticercoids (HDC)) which is directly given to humans. An example is the development of the cysticercoid stage of Hymenolepis diminuta. In this method, feces of the definitive host species (most commonly a rat) containing the ova of H. diminuta is cycled repeatedly using floatation solutions to remove them from the feces. When a relatively clean preparation of the ova is obtained they can be incubated in media to initiate fungal growth. Then washed in a solution containing one or more antifungal and antibacterial agents such as amphotericin and streptomycin. Then the ova are swirled in a solution containing small glass beads to crack the shell. The solution can then be filtered to remove the shells. The remaining portion of the ova are then placed in a solution to mature into the cysticercoid stage. This solution can be made up of one of the various standard off the shelf cell line media culture such as SF 900 from Life technologies. This solution can be supplemented with fetal bovine serum.

In one embodiment, rat feces can be obtained that contains Hymenolepis diminuta ova (HDO). The HDO is then washed to remove rat feces. The washed HDO can be sterilized. The HDO can then be incubated with fungi and then cleansed with antifungal before the HDO shells are cracked. Alternatively, the HDO shells can be cracked and removed before incubated with fungi and cleansed with antifungal. For example, the HDO is washed and swirled with glass beads to break the HDO shell, then the HDO without shells are incubated in the presence of fungi so that the fungus spores bloom. The incubation with fungi can result in the fungus spores germinating. Then, the fungi can be killed with an antifungal agent, and optionally antibacterial agent, which can result in a substantially sterile solution having the HDO. The HDO without shells are then incubated in cell culture media until converting into the cysticercoids—HDC. The HDC can then be isolated from the cell culture media. The isolated HDC can then be introduced into a consumable composition, such as the shipping solution described herein that is suitable for human consumption via oral administration.

In one embodiment, the cell culture media used to incubate the HDO to HDC can be Schneider's Drosophila Media, which optionally can be supplemented with serum, such as fetal bovine serum. However, other cell culture media can be used. The media can include an antifungal, and optionally an antibiotic.

In one embodiment, a method of manufacturing a consumable parasitic composition can include: obtaining a live parasite from an in vitro cell culture; and combining the live parasite with a consumable composition to provide the consumable parasitic composition, the consumable composition having nutrients to maintain the live parasite. In one aspect, the parasite includes Hymenolepis diminuta cysticercoids (HDC); however other cysticercoids of other species can be used.

In one embodiment, the method of manufacture can include preparing the consumable composition to include an antifungal, a plurality of different amino acids, a plurality of salts, a saccharide or polysaccharide, a plurality of different organic acids, and yeast extract. In one aspect, preparing the consumable composition can include one or more of the following: the antifungal is amphotericin B; the plurality of salts include two or more of NaCl, KCl, MgCl₂, NaH₂PO₄, or combinations thereof; the saccharide or polysaccharide include Trehalose, Glucose, or combinations thereof; or the plurality of different organic acids include alpha ketoglutaric acid, Fumaric acid, Malic acid, citric acid, succinic acid, or combinations thereof. In one aspect, the method can include obtaining the plurality of different amino acids to include: Alanine; Arginine; Aspartic acid; Cystine; Glutamic acid; Glycine; Histidine; Isoleucine; Leucine; Lysine; Methionine; Phenylalanine; Proline; Serine; Threonine; Tryptophan; Tyrosine; Valine; and Glutamine.

In one embodiment, the method of manufacture can include: obtaining the parasite to include Hymenolepis diminuta ova (HDO); maturing the HDO to Hymenolepis diminuta cysticercoids (HDC) in the in vitro cell culture; and collecting the HDC as the live parasite.

In one embodiment, the method of manufacture can include: incubating the HDO in cell growth media and fungi so as to initiate fungal growth; washing the HDO with antifungal; cracking shells of the HDO; and removing the cracked HDO shells. In one aspect, the washing of the HDO with antifungal occurs before the cracking of the HDO shells. In one aspect, the cracking of the HDO shells occurs before washing the HDO with antifungal.

In one embodiment, the method of manufacture includes: obtaining rat feces having the HDO; and obtaining cleansed HDO from the rat feces. A number of washing steps can be used to cleanse the HDO, such as typical washing or float washing so as to remove rat feces from the HDO.

In one embodiment, the method of manufacture can include: obtaining the parasite to include Hymenolepis diminuta ova (HDO) in rat feces; obtaining cleansed HDO from the rat feces; incubating the HDO in cell growth media and fungi so as to initiate fungal growth; washing the HDO with antifungal; cracking shells of the HDO; removing the cracked HDO shells; maturing the HDO to Hymenolepis diminuta cysticercoids (HDC) in the in vitro cell culture; and collecting the HDC as the live parasite.

In one embodiment, the method of manufacture can include obtaining the cleansed HDO includes repeatedly cycling flotation solutions to remove the HDO from the rat feces so that the HDO is sufficiently cleansed; incubating the HDO in cell growth media and fungi and bacteria so as to initiate fungal growth and bacterial growth; washing the HDO with antifungal and antibacterial; cracking the shells with hard beads (e.g., glass) in a swirled solution; filtering the cracked shells for removal of the shells; removing the HDC from cell culture media of the in vitro cell culture; and combining the HDC from the in vitro cell culture with the consumable composition that includes an antifungal, a plurality of different amino acids, a plurality of salts, a saccharide or polysaccharide, a plurality of different organic acids, and yeast extract. In one aspect, the consumable composition includes one or more of the following: the antifungal is amphotericin B; the plurality of salts include two or more of NaCl, KCl, MgCl₂, NaH₂PO₄, or combinations thereof; the saccharide or polysaccharide include Trehalose, Glucose, or combinations thereof; or the plurality of different organic acids include alpha ketoglutaric acid, Fumaric acid, Malic acid, citric acid, succinic acid, or combinations thereof.

In one embodiment, a consumable parasitic composition can include: a live Hymenolepis diminuta cysticercoids (HDC) raised from in vitro cell cultured Hymenolepis diminuta ova (HDO); a consumable composition having the live HDC, the consumable composition having nutrients to maintain the live HDC and an antimicrobial. In one aspect, the consumable composition includes an antifungal as the antimicrobial, a plurality of different amino acids, a plurality of salts, a saccharide or polysaccharide, a plurality of different organic acids, and yeast extract. In one aspect, the consumable composition includes: the antifungal is amphotericin B; the plurality of salts include two or more of NaCl, KCl, MgCl₂, NaH₂PO₄, or combinations thereof; the saccharide or polysaccharide include Trehalose, Glucose, or combinations thereof; and the plurality of different organic acids include alpha ketoglutaric acid, Fumaric acid, Malic acid, citric acid, succinic acid, or combinations thereof.

In one embodiment, a method of in vitro culturing Hymenolepis diminuta can include: obtaining rat feces having Hymenolepis diminuta ova (HDO); obtaining cleansed HDO from the rat feces; maturing the HDO to Hymenolepis diminuta cysticercoids (HDC) in the in vitro cell culture; and collecting the HDC from the in vitro cell culture. In one aspect, the method can also include: incubating the HDO in cell growth media and fungi so as to initiate fungal growth; washing the HDO with antifungal; cracking shells of the HDO; and removing the cracked HDO shells. In one aspect, the method can also include: obtaining the cleansed HDO includes repeatedly cycling flotation solutions to remove the HDO from the rat feces so that the HDO is sufficiently cleansed; incubating the HDO in cell growth media and fungi and bacteria so as to initiate fungal growth and bacterial growth; washing the HDO with antifungal and antibacterial; cracking the shells with hard beads in a swirled solution; filtering the cracked shells for removal; and removing the HDC from cell culture media of the in vitro cell culture. In one aspect, the method can also include: obtaining a rat; administering the HDC to the rat; and collecting rat feces from the rat, the rat feces having the HDO. A rat already having Hymenolepis diminuta such that the rat feces has HDO can be obtained.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, a method can include: obtaining a rat, cat or dog; administering the HDC or DCO to the rat, cat or dog; and collecting feces from the animal, the feces having the HDO or DCO. In one aspect, the method includes: obtaining a rat; administering the HDC to the rat; and collecting feces from the animal, the feces having the HDO. In one aspect, the method includes: obtaining a cat or dog; administering the DCO to the cat or dog; and collecting feces from the animal, the feces having the DCO. In one aspect, the method includes: obtaining rat feces having the HDO; and obtaining cleansed HDO from the feces. In one aspect, the method includes: obtaining cat or dog feces having the DCO; and obtaining cleansed DCO from the feces.

In one embodiment, the method can be employed without cracking the DCO, and thereby the DCO can be matured into DCC without cracking.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

References: US Pharmacopeia 2011 chapter 1111 and 2023, which is incorporated herein by specific reference in its entirety; Canadian Journal of Zoology, 1994, 72(4): 618-623, 10.1139/z94-084; The Biology of the Tapeworm Hymenolepis diminuta, 1980, 427-432

1. A method of manufacturing a consumable parasitic composition, the method comprising:
obtaining a live parasite from an in vitro cell culture; and
combining the live parasite with a consumable composition to provide the consumable parasitic composition, the consumable composition having nutrients to maintain the live parasite.

2. The method of claim 1, wherein the parasite includes Hymenolepis diminuta cysticercoids (HDC) or Dipylidium caninum cysticercoids (DCC).

3. The method of claim 2, further comprising preparing the consumable composition to include an antifungal, a plurality of different amino acids, a plurality of salts, a saccharide or polysaccharide, a plurality of different organic acids, and yeast extract.

4. The method of claim 3, further comprising preparing the consumable composition to include one or more of the following:
the antifungal is amphotericin B;
the plurality of salts include two or more of NaCl, KCl, MgCl₂, NaH₂PO₄, or combinations thereof;
the saccharide or polysaccharide include Trehalose, Glucose, or combinations thereof; or
the plurality of different organic acids include alpha ketoglutaric acid, Fumaric acid, Malic acid, citric acid, succinic acid, or combinations thereof.

5. The method of claim 4, further comprising obtaining the plurality of different amino acids to include: Alanine; Arginine; Aspartic acid; Cystine; Glutamic acid; Glycine; Histidine; Isoleucine; Leucine; Lysine; Methionine; Phenylalanine; Proline; Serine; Threonine; Tryptophan; Tyrosine; Valine; and Glutamine.

6. The method of claim 1, further comprising:
obtaining the parasite to include Hymenolepis diminuta ova (HDO) or Dipylidium caninum ova (DCO);
maturing the HDO or DCO to Hymenolepis diminuta cysticercoids (HDC) or Dipylidium caninum cysticercoids (DCC) in the in vitro cell culture; and
collecting the HDC or DCC as the live parasite.

7. The method of claim 6, further comprising:
incubating the HDO or DCO in cell growth media and fungi so as to initiate fungal growth;
washing the HDO or DCO with antifungal;
cracking shells of the HDO or optionally cracking the shells of the DCO; and
optionally removing the cracked HDO or DCO shells.

8. The method of claim 7, further comprising:
obtaining rat, cat, or dog feces having the HDO or DCO; and
obtaining cleansed HDO or DCO from the feces.

9. The method of claim 7, wherein the washing the HDO or DCO with antifungal occurs before the cracking of the HDO or DCO shells.

10. The method of claim 7, wherein cracking of the HDO or DCO shells occurs before washing the DCO or HDO with antifungal.

11. The method of claim 1, further comprising:
obtaining the parasite to include Hymenolepis diminuta ova (HDO) or Dipylidium caninum ova (DCO) in feces;
obtaining cleansed HDO or DCO from the feces;
incubating the HDO or DCO in cell growth media and fungi so as to initiate fungal growth;
washing the HDO or DCO with antifungal;
cracking shells of the HDO or optionally cracking shells of the DCO;
optionally removing the cracked HDO or DCO shells;
maturing the HDO to Hymenolepis diminuta cysticercoids (HDC) or DCO to Dipylidium caninum cysticercoids (DCC) in the in vitro cell culture; and
collecting the HDC or DCO as the live parasite.

12. The method of claim 11, comprising:
obtaining the cleansed HDO or DCO includes repeatedly cycling flotation solutions to remove the HDO or DCO from the feces so that the HDO or DCO is sufficiently cleansed;
incubating the HDO or DCO in cell growth media and fungi and bacteria so as to initiate fungal growth and bacterial growth;
washing the HDO or DCO with antifungal and antibacterial;
cracking the shells with hard beads in a swirled solution;
optionally filtering the cracked shells for removal;
removing the HDC or DCC from cell culture media of the in vitro cell culture; and
combining the HDC or DCC from the in vitro cell culture with the consumable composition that includes an antifungal, a plurality of different amino acids, a plurality of salts, a saccharide or polysaccharide, a plurality of different organic acids, and yeast extract.

13. The method of claim 12, wherein the consumable composition includes one or more of the following:
the antifungal is amphotericin B;
the plurality of salts include two or more of NaCl, KCl, MgCl₂, NaH₂PO₄, or combinations thereof;
the saccharide or polysaccharide include Trehalose, Glucose, or combinations thereof; or
the plurality of different organic acids include alpha ketoglutaric acid, Fumaric acid, Malic acid, citric acid, succinic acid, or combinations thereof.

14. A consumable parasitic composition comprising:
a live Hymenolepis diminuta cysticercoids (HDC) or Dipylidium caninum cysticercoids (DCC) raised from in vitro cell cultured Hymenolepis diminuta ova (HDO) or Dipylidium caninum ova (DCO); and
a consumable composition having the live HDC or DCC, the consumable composition having nutrients to maintain the live HDC or DCC and an antimicrobial.

15. The composition of claim 14, wherein the consumable composition includes an antifungal as the antimicrobial, a plurality of different amino acids, a plurality of salts, a saccharide or polysaccharide, a plurality of different organic acids, and yeast extract.

16. The composition of claim 15, wherein the consumable composition includes:
the antifungal is amphotericin B;
the plurality of salts include two or more of NaCl, KCl, MgCl₂, NaH₂PO₄, or combinations thereof;
the saccharide or polysaccharide include Trehalose, Glucose, or combinations thereof; and
the plurality of different organic acids include alpha ketoglutaric acid, Fumaric acid, Malic acid, citric acid, succinic acid, or combinations thereof.

17. A method of in vitro culturing Hymenolepis diminuta, the method comprising:
obtaining feces having Hymenolepis diminuta ova (HDO) or Dipylidium caninum ova (DCO);
obtaining cleansed HDO or DCO from the feces;
maturing the HDO to Hymenolepis diminuta cysticercoids (HDC) or DCC to Dipylidium caninum cysticercoids (DCC) in the in vitro cell culture; and
collecting the HDC or DCC from the in vitro cell culture.

18. The method of claim 17, further comprising:
incubating the HDO or DCO in cell growth media and fungi so as to initiate fungal growth;
washing the HDO or DCO with antifungal;
cracking shells of the HDO or optionally cracking shells of the DCO; and
optionally removing the cracked HDO or DCO shells.

19. The method of claim 18, further comprising:
obtaining the cleansed HDO or DCO includes repeatedly cycling flotation solutions to remove the HDO or DCO from the feces so that the HDO or DCO is sufficiently cleansed;
incubating the HDO or DCO in cell growth media and fungi and bacteria so as to initiate fungal growth and bacterial growth;
washing the HDO or DCO with antifungal and antibacterial;
cracking the shells with hard beads in a swirled solution;
optionally filtering the cracked shells for removal; and
removing the HDC or DCO from cell culture media of the in vitro cell culture.

20. The method of claim 19, further comprising:
obtaining a rat, cat or dog;
administering the HDC or DCO to the rat, cat or dog; and
collecting feces from the animal, the feces having the HDO or DCO.
